(19) United States
(12) Patent Application Publication
(10) Pub. No.: US 2025/0040691 A1
(43) Pub. Date: Feb. 6, 2025

(54) Mechanical Retainer Brush with Interchangeable Bristle Heads

(71) Applicant: Nikki Yamamoto, San Gabriel, CA (US)

(72) Inventor: Nikki Yamamoto, San Gabriel, CA (US)

(21) Appl. No.: 18/738,655

(22) Filed: Jun. 10, 2024

Related U.S. Application Data
(63) Continuation of application No. 63/530,067, filed on Aug. 1, 2023.

(51) Int. Cl. A46B 13/04; A46B 5/00; A61L 2/10

(52) U.S. Cl. CPC A46B 13/04; A46B 5/0095; A61L 2/10; A46B 2200/3073

(57) ABSTRACT

This invention is a mechanical device that allows for cleaning and sanitation of retainers through brush and cleaning liquid or water propelling technology.

### FIELD OF INVENTION

This invention relates to retainer cleaning equipment.

### BACKGROUND

In the present day, people of all ages utilize retainers. However, because there has been no explicitly proper way of cleansing them, many have faced difficulties in keeping them clean. Thus, customers have been divided into two. One group uses their toothbrushes to scrub away residue and calcium buildup, unable to sanitize away bacteria and barely scraping the surface. Further, some use toothpaste to clean the retainers, leading to further damage. The other group drowns the retainers in cleansing fluid, extirpating most of the bacteria but leaving occasional buildup. However, some cleaning fluid casing makes it difficult to carry around.

### PROBLEMS WITH ORDINARY CLEANING EQUIPMENT

The closest cleaning technology to this invention are non-mechanical toothbrushes shaped in a way more suited to cleaning retainers. Due to its soft bristles, many customers did not prefer the product. They stated that it did not work well or at all. Another method on the market is UV retainer cases. There has been some uncertainty of cleaning retainers with UV rays, as it may impact its structural strength overtime. Lastly, placing retainers in cleaning fluid for long durations could cause some sensitivity among certain individuals. Because retainers are drenched in cleaning liquids and employ hydrogen peroxide, some have reported it burning their tongue and stinging their gums.

Therefore there lies a need for retainers to be physically brushed in order to extensively rid of plaque and calcium buildup. There also exists a need for cleaning fluid or higher pressure water to expunge bacteria. Finally, there is a need for the equipment to be easy to use, durable, and efficient for traveling.

### PROPOSED SOLUTIONS FOR CURRENT PRODUCTS ON THE MARKET

1. Retainer brushes. These brushes are strikingly similar to the average toothbrush, aside from its taller bristles concentrated in a smaller area of the head. One of its biggest drawbacks are how soft and flexible the brush is. This enables it to only slightly clean the surface of the retainer, not able to vigorously scrub the areas in need of more concentrated sanitation. Thus, the bristles should be stubbier and of a thicker material. Instead of its conventionally thin design, the diameter of each strand can be thickened to prevent unnecessary flimsiness and flexibility.

2. UV retainer cases. Many people prefer this form of sterilization as it is the most convenient and hands off. However, because of its possible detriment to the retainer plastic itself, it may be better to reserve the UV rays for other sanitary uses. Instead, those who are not inclined to use retainer brushes can opt for a cleaning fluid. Cleaning fluid is generally less time consuming than dry-brushing the retainers. However, due to possible sensitivity issues with the chemicals in the liquid, consumers have the option of selecting solutions more suitable for their comfort.

3. Engulfing in cleaning fluid. If retainers are placed in cleaning fluid for too long, the chemicals stick onto the retainer and give it a chemical aftertaste. Furthermore, some children who use retainers pour the cleansing liquid into warm or hot water, bending the retainers to a point where it can no longer be used. There should be clear instructions when utilizing cleaning liquid, and retainers should not be drenched for more than the recommended time. Rather than the conventional way of placing it in a cup and sitting there, the retainers can be washed over with the fluid instead.

### SOLUTION AND BRIEF DESCRIPTION

This invention is a mechanical retainer cleaning brush with a sleek design. Equipped with brush and liquid-propelling technology, it solves for many of the previously stated challenges faced by current consumers. The bristles are designed with various shapes and sizes to be customizable to each individual, allowing for the unique curves and crevices of each person's retainers to be thoroughly cleaned. This brush encompasses a 360° rotating technology, allowing for full coverage. The brush head propels a cleansing liquid of your choice onto the area the bristles are cleaning, for both a physical and chemical purification. The UV lights inside the cap of this invention enables self-cleaning of the brushes, consistently keeping the device sanitary for next use. Additionally, there are different levels of liquid propelling intensity from hard to soft by the push of a button. Easy to clean out the remaining liquid in the bottle, simply unscrew the head and rinse. Durable material makes it a long-term investment.

### THE OBJECTIVE

The objective of this device is to not only make the brushing process easier and spread over a larger area, but also to use cleaning fluid to break down remaining residue all at once. This way there is a prevention of multi-step solutions that inconvenience many, to the point of becoming negligent of retainer cleanliness.

### FUTURE MANUFACTURING OPTIONS

Depending upon the manufacturer and their methods, the bottle can possibly be made from bamboo to stay environmentally friendly. The bamboo would be cut, shaped, and sealed to make it capable of holding liquids. The body and cap will be produced in the same fashion, with the cap having strips of UV light securely attached to sanitize the brush head. The tube attaching the brush head to the bottle will be made of possibly plastic, along with the bristles of possibly nylon fibers (as a conventional toothbrush is) with a slightly larger diameter and shorter height. An indent will be pressed into the bottom of the tube to create a clicking contraption that attaches it to the bottle by twisting to one side. The hole on the plate that clicks with the tube will be created to rotate at a fast pace, with the surrounding plate unmoving. The sides of the plate contain ridges (made with possibly magnetic materials) to attach with the cap. This enables secure closing. To remove the cap and clean the inside of the bottle, slowly unscrew it. Lastly, for the on and off button and liquid propelling speed, two buttons with a rubber texture will be placed on the outside bottom of the bottle, reserving a small space around the diameter of the inside of the bottle for necessary wires to function. Ensure that the cap clicks onto the ridges of the bottle, securing the device in a closed position.

There are various ways a manufacturer may decide to produce the product, acknowledging that they will create it based upon experience and most optimal practices to determine what needs alterations.

### BRIEF DESCRIPTIONS OF DRAWINGS

FIG. 1. A front and side view of the retainer cleaning device.

1A. Device cap.

1B. Button for maneuvering speed of propelled liquid.

1C. On and off button.

1D. Device bottle (body).

1E. Top and bottom view (the same).

FIG. 2. Device without cap, image of the brush head.

2A. Brush head.

2B. Liquid transfer tube.

2C. Ridged cap, unscrewable top to reveal bottle inside.

FIG. 3. Parts of the device separated.

3A. UV light spread out, but surrounds the inside of the cap.

3B. How the UV light looks inside.

3C. Internal ridges on the cap.

3D. Hole for connecting the brush tube.

3E. End of the tube.

3F. Bottle inside body, holding liquid.

FIG. 4. Varying brush heads.

4A. Extra large-sized brush.

4B. Large-sized brush.

4C. Medium-sized brush.

4D. Small-sized brush.

4E. Extra small-sized brush.

### DETAILED DESCRIPTION OF THE INVENTION

The present disclosure is an exemplification of the device, and is not meant to restrain or limit it to specific structural designs as illustrated or described.

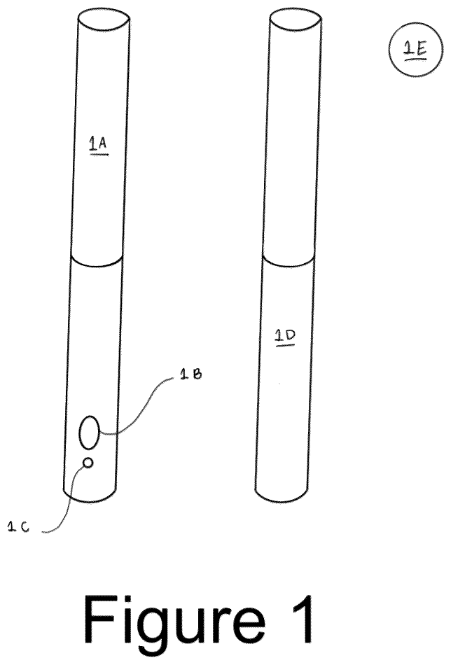

The first aspect to be focused on is the outward appearance (FIG. 1). The cap (1A) and body (1D) are of similar dimensions and possibly bamboo material. The top and bottom of the device (1E) are the same, as the top and bottom are of equal measurements. The design should be clean and minimalistic. Towards the bottom of the body are two buttons; The larger one is meant for alternating the speed of the propelled liquid (1B). There are three main settings: 1, 2, 3. The first is the softest, with the third being the roughest. Based on the needs of the consumer, these settings can be thus adjusted. The button below the above described is the on and off button, (1C) one click will turn it on or off. Both buttons could be made of a stiffer rubber or silicone, so that it is a little harder to press. This will prevent any accidental pressure from starting the device.

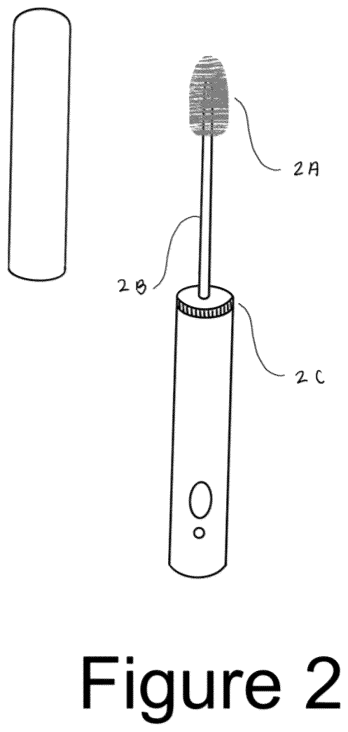

Upon taking off the cap (FIG. 2), the brush head and brush tube are displayed. The brush head (2A) will be comprised of shorter, stiffer toothbrush material that can easily scrape off buildup and bacteria. The 360° rotating technology allows this brush to gyrate and create speed. The tube (2B) connects the brush head to the main body, transporting water or preferred cleaning liquid from the bottle compartment to the tip of the brush. The liquid will squirt out similar to a jet, and work alongside the bristles to sterilize. This tube would be made out of sturdy, BPA-free plastic to ensure durability. Can be easily cleaned with a straw cleaner. The ridges on the top sides of the body (2C) have two essential purposes: The first is to allow secure closing with the cap, which also possesses similar ridges (3C) that snap together upon impact (through magnetism). The second purpose is to allow access to the inside of the bottle, simply by unscrewing it. The ridges allow for easier grasp, though it should take effort to turn for extra security.

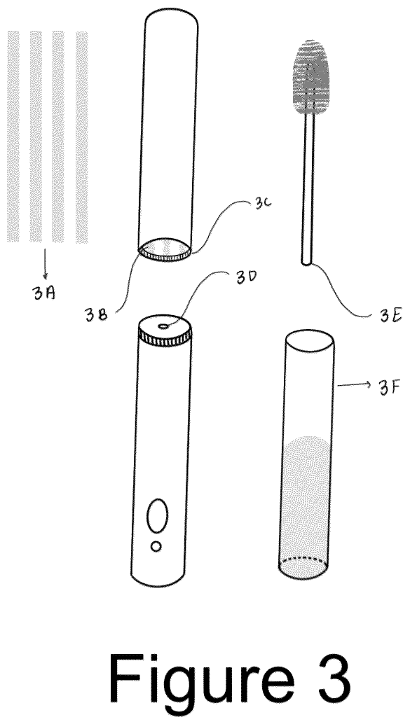

In FIG. 3, the anatomy of the cap is shown. All around the inner area there are attached UV lights (3B) aligned in rows to automatically clean the brush head and tube. The cross-section diagram of the UV rays are shown for a closer look (3A). This enables repeated usage of the clean brush over short amounts of time. The top of the bottle plate area is a hole (3D) for placing the brush tube. An indent will be present on the bottom of the brush tube (3E) (unless there is another way to firmly attach the two entities) that will “click” with the plate. Lastly, the internal structure of the bottle (3F) displays the waterproof interior meant to hold cleaning fluid or water. This compartment can be easily purified with soap and water, and simply pour in cleaning fluid and screw the top back on to reuse.

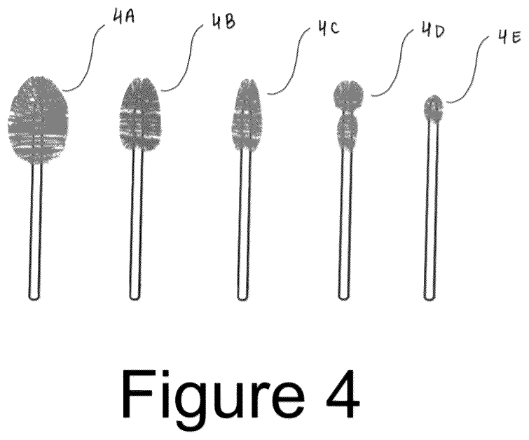

FIG. 4 displays the multiple brush heads possibly available. As retainers come in all shapes and sizes, having brushes of different structures guarantees a customizable experience. The current sizes are extra-large (4A), large (4B), medium (4C), small (4D), and extra-small (4E). Extra small could be utilized for smaller ridges, especially around the molars, while the extra large could be employed for general coverage cleansing.

### CONCLUSION

It can be concluded that this new invention will act as an improved method of retainer sanitation. Its combination of both brushing and cleaning solution technology enables a thorough, one-step process to alleviate the concerns of many consumers. This invention includes a UV light cap, bottle, “click” in tube with attached brush head, mechanisms to release water from the head to the bristles and retainer, as well as all else provided in the descriptions and diagrams.

1. A retainer cleaning device comprising:
a body with internal room to store cleansing liquids;
a replaceable brush that moves mechanically removably secured to the top of said body:
and a cap with UV light removably secured to the top of said body covering the said replaceable brush.
